(19) United States
(12) Patent Application Publication
(10) Pub. No.: US 2021/0128691 A1
(43) Pub. Date: May 6, 2021

(54) EXTENDED RELEASE L-TRI-IODOTHYRONINE AMELIORATES THE PATHOPHYSIOLOGY OF THE MATERNAL PRE-ECLAMPSIA SYNDROME

(71) Applicant: JOSEPH MICHAEL ABRAMOWITZ, SAN DIEGO, CA (US)

(72) Inventor: JOSEPH MICHAEL ABRAMOWITZ, SAN DIEGO, CA (US)

(21) Appl. No.: 17/086,963

(22) Filed: Nov. 2, 2020

Related U.S. Application Data
(60) Provisional application No. 62/929,864, filed on Nov. 2, 2019.

(51) Int. Cl. A61K 38/22; A61K 9/00; A61P 15/00

(52) U.S. Cl. CPC A61K 38/22; A61P 15/00; A61K 9/0002

(57) ABSTRACT

The present disclosure describes a method for treatment of pre-eclampsia including treatment with Extended Release L-triiodothyronine (T3) and thyroxine (T4). In an alternate composition and method Extended Release T3, T4, and at least one phosphodiesterase (PDE) inhibitor, being a combined 3/4 or a PDE inhibitor, or a combination of these PDE inhibitors may be used for treatment.

### RELATED APPLICATIONS

This application claims the benefit of priority of U.S. Provisional Application Ser. No. 62,929,864 filed Nov. 2, 2019 of common inventorship and which is hereby incorporated by reference in its' entirety.

### FIELD OF THE INVENTION

The present invention relates to the condition of pre-eclampsia, also known as ‘pre-eclampsia/eclampsia’, ‘toxemia of pregnancy’ and ‘pregnancy induced hypertension’ and more particularly to methods of preventing, stabilizing or reversing the condition.

### BACKGROUND

Pre-eclampsia (PE) has been an enigma of the obstetric experience for centuries. Hypertensive disorders of pregnancy account for 10-20% of maternal deaths worldwide. It is estimated that globally, each year, at least 50,000 to 100,000 women die from this condition. It is also estimated that globally at least 500,000 newborns suffer serious consequences. Many of these newborns suffer perinatal mortality or are afflicted with chronic disease, in part consequent to extreme prematurity, for the rest of their lives. PE is considered by many to be a uniquely human disease, although there are rare reports of the condition in higher non-human primates. PE is generally studied in the laboratory in rodent models of the condition.

PE is more common in first pregnancies where the incidence is in excess of 10%. Across all pregnancies, the incidence of PE is around 5%. Other risk factors include a positive family history, a pre-existing diagnosis of hypertension, pre-existing renal disease, diabetes and obesity. Trophoblastic disease and PE in a prior pregnancy are also risk factors. A history of early onset PE in a prior pregnancy is associated with a risk of PE in a subsequent pregnancy which is in excess of 30%. In the event that there is a change in paternity, the PE risk with the new father is almost as high as in first pregnancies. This finding resonates with the hypothesis described below in the present disclosure. The cause of PE remains unknown. Mitochondrial inheritance, genomic imprinting, inflammation, autoimmunity and environmental factors are believed to contribute to the expression of the disease. Further, women who have been afflicted with PE in a prior pregnancy carry a lifetime increased risk of cardiovascular disease, including hypertension, ischemic heart disease and stroke. Women with a history of PE beginning in the second trimester are at increased risk for syndromes of insulin resistance and lipid abnormalities in later life. These epidemiologic findings suggest that, in a significant number of women with a history of PE, the damage sustained by the maternal metabolic infrastructure during the index pregnancy is permanent.

There are two pathologic hallmarks of PE reflecting the abnormal placentation found in the condition. The first is cytotrophoblast pseudovasculogenesis. Here cytotrophoblast cells invade maternal blood vessels and assume an endothelial phenotype. The second is acute atherosis where the histology shows fat laden macrophages and perinuclear infiltrates which are found in arteries not invaded by trophoblastic cells. These elements of pathologic placentation have been a focus of researchers but their significance remains uncertain.

In regard to thyroid hormone (TH) metabolism, the past few decades have heralded much research into and an understanding of the iodothyronine deiodinase (DI) enzymes whose job it is, in space and time, to defend the optimum balance of TH. The balance of thyroid hormone (BoTH) is defined as a space and time dependent phenomenon whereby a precise degree of TH activation or inactivation is called for and achieved. Peripheral blood measurement of thyroid stimulating hormone (TSH), L-tri-iodothyronine (T3) and L-thyroxine (T4) are examples of the so-called thyroid function tests and they are tests reflecting thyroid hormone kinetics. The term kinetics refers to what the body does to thyroid hormone, regulating its' manufacture and transport. These tests tell little about the adequacy of the executive function of TH in the target tissues, which lies in the domain of TH dynamics. The term dynamics refers to what TH does to the body. There are no blood tests in current clinical usage which can confirm adequacy of these executive functions.

Presently there are no available treatment modalities for PE which can prevent, stabilize or reverse the condition. Intravenous magnesium sulfate can stabilize the patient for a safer expedited delivery but this modality cannot prolong the pregnancy to term if PE precipitates a preterm emergency. Although much more is presently known about PE than was known centuries ago, the risks to mother and fetus have changed little.

### SUMMARY

Pre-eclampsia (PE) is caused by a paternal imprinted gene (PIG) in a genetically susceptible mother. The paternal gene or multi-gene imprinted domain acts from its vantage in the placenta and sets in motion an attack on elements of maternal metabolism. This attack on the molecular biology of the mother is directed towards the goal of giving the fetus an advantage in post-natal life. This scenario is consistent with the core biologic-philosophic theory of imprinted genes, the ‘parental conflict hypothesis’.

The patho-physiology of pre-eclampsia is complex. It is generally accepted that a deficit of the gasotransmitters NO and CO in the mother is the primary known upstream anomaly in PE. Although much is known regarding the enzymatic reactions producing nitric oxide (NO) and carbon monoxide (CO), the agent initiating the deficits of NO and CO is unknown. The art of the present disclosure claims to know the identity of the initiator, as will be explained below. There are numerous intermediate biochemical aberrations occurring downstream from the gasotransmitter aberrations referenced, including involvement of: tumor necrosis factor-alpha, phosphatidyl inositol-glycan biosynthesis class F protein, soluble cell adhesion molecule E-selectin, soluble fms-like tyrosine kinase-1, soluble endoglin, TH1 cytokine and Endothelin 1. These aberrations are presumed secondary to the pathophysiology described in FIG. 7A and FIG. 7B and they are presumed corrected by the method of the present disclosure, described in FIG. 8A and FIG. 8B. Consequently they will not be further referenced.

Anomalies in TH dynamics may exist in the face of normal TSH and free T4 levels. Thus anomalies may exist which impact what TH does to the body without being reflected in routinely ordered thyroid blood tests. Controversy exists over which TH replacement therapy is best, whether it is T4 monotherapy or the T3/T4 combination exemplified by desiccated thyroid (DT). The current generation of thyroidologists and their expert committees, both in the United States and in Europe, have voiced concerns regarding T3/T4 combination therapy: (i) They believe correctly that immediate release T3 administration results in absorption spikes in plasma levels of T3 which are supra-physiologic and which put the patient at risk for cardiac arrhythmias; (ii): They point out that batches of animal-sourced DT have inconsistent ratios of T4 to T3; (iii): They allege that the ratio of T4 to T3 found in animal-sourced DT is not the same as the ratio found in humans. Consequently, these expert committees recommend that T4 monotherapy is the standard of care for TH replacement therapy. What is omitted here is that T4 monotherapy results in an increased ratio of T4:T3 in the plasma of the recipient of the pharmaceutical, a phenomenon which is not physiologic and which has been questioned. Elevated T4 blood levels are known to lead to ubiquitination of D1 type 2 (D2) {1}. While this ubiquitination reaction is reversible, it inactivates the enzyme, slowing the rate of the activation reaction, which converts T4 to T3.

Further, the efficacy of T4 monotherapy depends upon the capacity of the organism to convert T4 to T3, a phenomenon necessitating normal function of D2. In cases of an inherited polymorphism of D2 and in cases of acquired conditions associated with endoplasmic reticulum (ER) stress (which is associated with reduced D2 activity) such as PE, type 2 diabetes mellitus and Alzheimers disease, the activation reaction executed by D2 is substantially reduced {2}. Teleologically the human organism has evolved with a strict mandate to protect itself from unwanted TH activation. This is evidenced by the manner in which TH is handled in the human embryo as well as in post-natal target tissues {3}. This phenomenon makes the human organism vulnerable to metabolic aberrations which jeopardize TH activation.

Over the past few decades it has become known that central to the downstream biochemical pathology in PE is the generation of suboptimal quantities of the gasotransmitters NO and CO {4}. Hydrogen sulfide (H2S), also known as ‘the third gasotransmitter’, presently lacks the clear role played by NO and CO in PE. While H2S is reduced in the placenta of the PE patient and while certain H2S synthetic enzymes are dysregulated in PE, the role of H2S in PE is by no means clear.

It is an object of the present disclosure to restore the production of normal quantities of the referenced gasotransmitters, NO and CO, and the method for achieving this is described hereinafter in the specification and claims. The referenced gasotransmitters are required for the generation of cyclic guanosine monophosphate (cGMP), the cyclic nucleotide, a cGMP sufficiency of which maintains vasodilatation. NO is generated by the nitric oxide synthase (NOS) reaction whereby one molecule of NO is generated by the enzyme from a molecule of diet-sourced L-arginine. The heme oxygenase (HO) reaction generates one molecule of CO and one molecule of biliverdin from a molecule of heme. H2S generation involves four enzymes: cystathionine gamma lyase (CSE); cystathionine beta synthase (CBS); 3-mercaptopyruvate sulfurtransferase (MST); cysteine aminotransferase (CAT). Phosphodiesterases constitute a class of enzymes which inactivate cyclic nucleotides. The roles played by PDEs in PE pathophysiology and the roles played by inhibitors of PDEs in the remedial aspects of the present disclosure are described in detail below. Heme oxygenase (HO) is a microsomal enzyme which has assumed a critical importance in the theory of the pathogenesis of PE. HO activity provides protective cellular effects through its' enzymatic actions. It has anti-complement effects and anti-oxidant effects. Via its' production of CO, it has anti-platelet and vasodilatory properties. Decreased HO activity has been found in placentas of PE patients {5}. Suboptimal HO activity in PE is presently suspected to be one of the prime causes of the aberrant placental vascular pattern seen in PE {5}. Decreased HO activity has also been found in maternal blood in PE {6}.

Central to the patho-physiology of PE, and responsible for the end result of endothelial damage, is a disruption of the ‘thyroid hormone chemopil’ (THC), previously uncharacterized and unpublished but explained below, leading to an aberration in the BoTH in the mother. As a consequence the metabolism of the mother suffers a unique form of cellular hypothyroidism, lacking classical clinical signs of hypothyroidism, one that is masked by the pregnant state. One of the chief consequences of this ‘masked hypothyroidism’ is a failure to sustain the generation of a sufficiency of the gasotransmitters NO and CO. This arises because the sub-threshold thyroid hormone activity is insufficient to maintain normal activity of the maternal enzymes NOS and HO, and is also insufficient to maintain adequate suppression of PDE activity. This leads to a sub-threshold level of cyclic guanosine monophosphate which in turn leads to a cascading effect of aberrations in protein kinase activity as well as in potassium and calcium channels, culminating in endothelial damage and vasoconstriction. Further the cellular hypothyroidism adversely affects sarco/endoplasmic reticulum calcium ATP-ase (SERCA) related proteins. This results in incomplete calcium reuptake in the sarcoplasmic reticulum. The residual calcium in the cytosol downregulates cyclic guanosine monophosphate and it also contributes to the vasoconstriction.

In the most serious and acute ante-partum form of pre-eclampsia, the ‘HELLP syndrome’ (hemolysis; elevated liver enzymes; low platelets), these effects lead to hypertension, micro-angiopathic hemolytic anemia, thrombocytopenia, renal damage with proteinuria and an ischemic hepatopathy with elevated liver enzymes. The effect of the PIG on the maternal metabolism is associated chiefly, but not exclusively, with the intermediary metabolism of thyroid hormone.

In PE there are two mechanisms resulting in cellular hypothyroidism. The first occurs as a consequence of the PIG attack on the THC of the mother. This causes inactivation, by ubiquitination, of D2 by upregulated sonic hedgehog signaling (the PIG also produces upregulation of iodothyronine deiodinase type 3 (D3) by transformational growth factor-beta). The second occurs due to ER stress resulting in a substantial reduction in the activation of thyroid hormone by D2.

T3 restores cellular euthyroidism and facilitates the production of a sufficiency of NO and CO, achieving the latter by upregulating both NOS and HO. T3 causes reduced activity of the PDE enzymes as part of its' adrenergic mandate to maintain a sufficiency of cyclic nucleotides. PDE inhibition, by TH, PDE inhibitors or both, contributes to the maximization of gasotransmitter production. T3 normalizes sarco/endoplasmic reticulum calcium ATP-ase function, via phospholamban and calsequestrin regulation, normalizing calcium reuptake. Because T3 is the active form of thyroid hormone it bypasses the impediment of the impaired D2 activity, occurring due to the PIG effect and to the ER stress effect.

The present disclosure outlines a method and formulation for preventing and/or treating PE with extended release T3 (ERT3) alone or with ERT3 and PDE inhibitors. When ERT3 therapy, presented in a pharmaceutical composition of the present disclosure, is initiated in the mother, to prevent pre-eclampsia, during the first or second trimester of pregnancy and continued to term; or initiated in the mother, to treat pre-eclampsia, at the time of diagnosis of PE and continued to term; the pharmaceutical of the present disclosure may lead to the generation of a sufficiency of the gasotransmitters NO and CO. The pharmaceutical of the present disclosure also may maintain the balance of intracellular calcium in endothelial tissues such that normal vascular reactivity and, in particular, vasodilatation is maintained. The administration of ERT3, with or without PDE inhibitors, may prevent, reverse or stabilize the clinical manifestations of the maternal PE syndrome. The combination of ERT3 with a PDE 3/4 inhibitor and/or a PDE 5 inhibitor may allow the polypharmaceutical to be efficacious at low doses of all components. It is anticipated that with the latter embodiment, ERT3 doses as low as 4-8 mcg/24 hours may be efficacious when used together with intermediate doses of the PDE inhibitors.

### BRIEF DESCRIPTION OF THE DRAWINGS

FIG. 1 is a thought experiment on PE, tracing the steps from problem to solution.

FIG. 2 delineates the Thyroid Hormone Chemopil.

FIG. 3 lists the Thyroid Hormone Dysregulation Syndromes identified.

FIG. 4 shows the attack on the maternal Thyroid Hormone Chemopil by the Paternal Imprinted Gene.

FIG. 5 is an overview of the maternal pathophysiology in pre-eclampsia.

FIG. 6A shows maternal endoplasmic reticulum stress in pre-eclampsia without T3 supplementation.

FIG. 6B shows maternal endoplasmic reticulum stress in pre-eclampsia with T3 supplementation.

FIG. 7A shows maternal pre-eclampsia pathophysiology leading to deficits in the formation of cGMP.

FIG. 7B shows maternal pre-eclampsia pathophysiology downstream from cGMP.

FIG. 8A shows corrected pre-eclampsia biochemistry, with reference to FIG. 7A, by the supplementation of T3 and PDE inhibition.

FIG. 8B shows corrected pre-eclampsia biochemistry, with reference to FIG. 7B, by the supplementation of T3 and PDE inhibition.

FIG. 9 shows effects of hypo- and hyperthyroidism on maternal immune and inflammatory responses.

Before explaining the embodiments of the present disclosure in detail, it is to be understood that the disclosure is not limited in its application to the details of the particular arrangement shown, since the invention is capable of other embodiments. Also, the terminology used herein is for the purpose of description and not of limitation.

### DETAILED DESCRIPTION

Pre-eclampsia (PE) is caused by a PIG in a gestation carried by a genetically susceptible mother. PE originates in the placenta where functional and structural abnormalities are evident in the first trimester. Clinically significant maternal pathophysiology begins in the second or third trimester, after the 20^{th} week of gestation. Because PE is a genetic disease, true prevention will require some form of genetic engineering which, arguably, is decades away. It is an object of the present disclosure to prevent, stabilize or reverse the maternal PE syndrome such that PE does not jeopardize the health of the mother. Thus, in the absence of another medical complication of pregnancy, or of an obstetric complication, the pregnancy may be sustained until term and a healthy full-term fetus may be delivered with a weight appropriate for gestational age.

The present disclosure does not knowingly address the abnormal placentation or its' consequences to the fetus. The fetal intrauterine growth retardation (IUGR) associated with PE may be prevented or ameliorated by the method of the present disclosure as it prevents, stabilizes or reverses the maternal

PE syndrome. This will be the subject of study. Most skilled in the art would opine that the primary placental pathology in PE is responsible for the IUGR. However, it is unknown if, and to what extent, the severity of the maternal PE syndrome aggravates the placental disease and worsens the fetal condition. Prior to the art of the present disclosure, there has not been an opportunity to remove the maternal PE syndrome as an independent variable and then to observe the outcome of the pregnancy as determined solely by placental factors. In the event that the method of the present disclosure ameliorates the maternal PE syndrome and improves the fetal outcome, then the abnormal placentation may not be of any consequence.

It has become apparent that deranged vasodilator gasotransmitter metabolism, primarily involving NO and CO plays a key role in the pathogenesis of the endothelial damage in PE. Although the physiologic generation of these gaso-transmitters is complex, as will be disclosed below, L-tri-iodothyronine, the active form of TH, plays a key role. As will also be disclosed below, the generation of a sufficiency of T3, in maternal tissues as well as the capacity for said T3 to fulfill its genomic and non-genomic mandates, is impaired in PE.

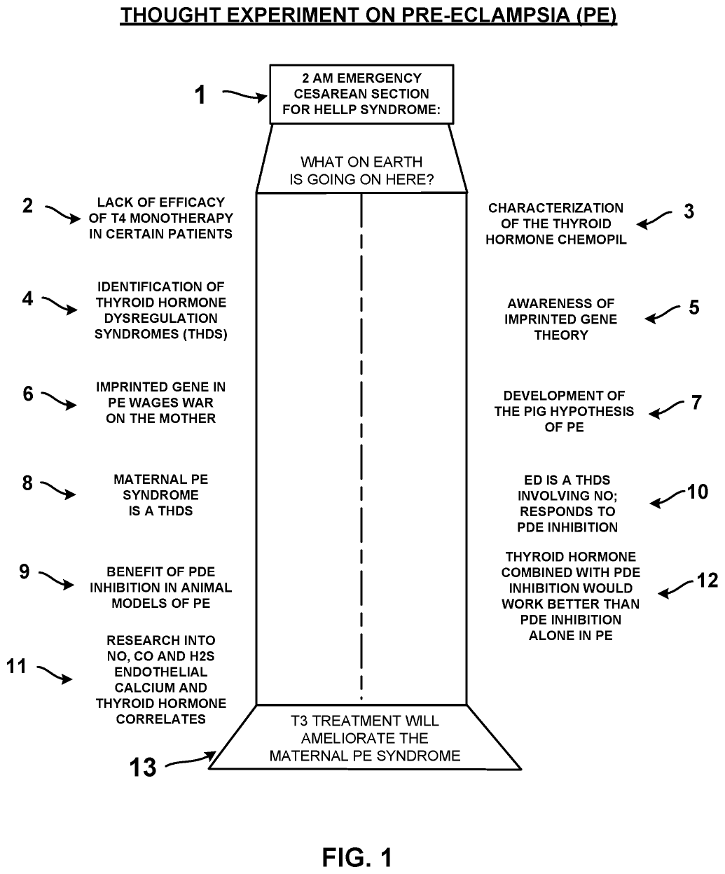

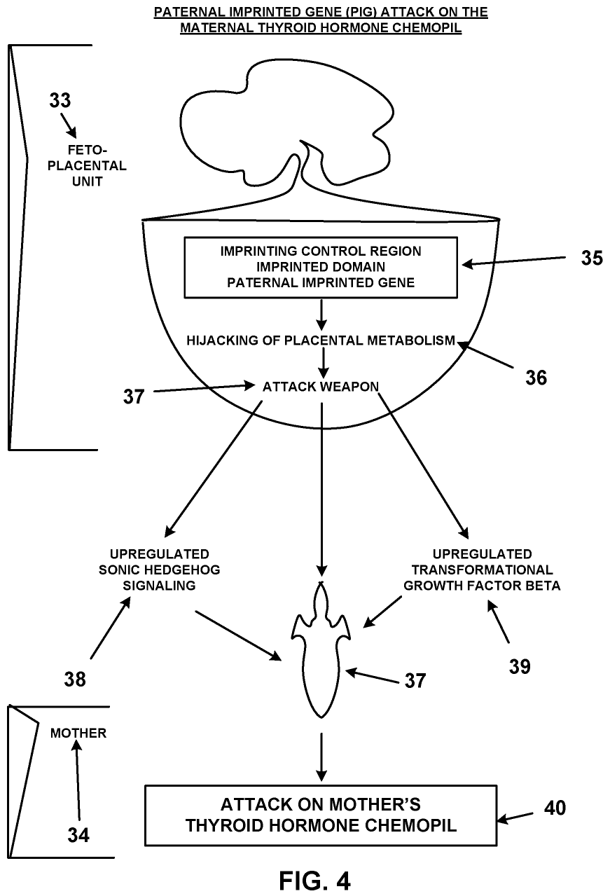

FIG. 1 presents a road map of a sequence of thought experiments, the path to an understanding the cause of, and remedy for, PE. Observations of the anomaly of PE were made during the administration of anesthesia for emergency cesarean section to expedite delivery of compromised fetuses in parturients with HELLP syndrome 1. This emergency scenario would generally repeat, often at around 2 am. The recurring question was “what on earth is going on here?” This question would resurface and haunt . . . for decades. Another anomaly was noted when treating patients with thyroid hormone replacement, when a suspicion developed that T4 monotherapy was associated with a lack of efficacy in certain patients 2, (about 15%) a phenomenon which, although suspected by some, has not been widely accepted. An analysis of the concept of the BoTH followed, which included study of the DI enzymes, TH receptors, membrane transporters, and what is known of signaling molecules. Although multiple components of this system had been identified, it had not been concretized in the form of a single entity. Such concretization might benefit understanding of thyroid physiology and its' application to pathophysiologic states. A name was developed for this molecular biologic infrastructure dedicated to maintaining the BoTH. This led to the characterization of the ‘Thyroid Hormone Chemopil’ (THC) 3. Concretization of the THC led to the identification of dozens of syndromes, not formally known to be associated with abnormal TH dynamics, which are related to THC aberrations. This led to designation of the term ‘Thyroid Hormone Dysregulation Syndromes’ (THDS) 4 which generally exist in the face of a normal TSH and a normal free T4. Next the imprinted gene theory 5 was addressed. An hypothesis was developed positing that imprinted gene in PE wages war on the mother 6, by directing an attack on the mothers' molecular biology. This was then termed the ‘Paternal Imprinted Gene (PIG) hypothesis’ of PE 7. The PIG gene complex directs its' attack on the maternal THC, as will be explained further in the narrative on FIG. 4. Thus the maternal PE syndrome is a THDS, as shown in FIG. 1, 8. Benefits of PDE inhibition in animal models of PE have been demonstrated 9. Erectile dysfunction (ED) in human males is a THDS, involving NO, responding to PDE inhibition 10, and may be responsive to T3. Prescription drug therapy for ED involves PDE inhibition. T3 has effects minimizing PDE activity as part of its' adrenergic mandate to maintain levels of cyclic nucleotides. Research into NO, CO and H2S generation and actions in PE and interacellular calcium balance reveals correlations with TH 11 actions. It was hypothesized that TH combined with PDE inhibitors would likely be more effective than PDE inhibition alone in the maternal PE syndrome 12. TH independently facilitates PDE inhibition {7}{8}, by unknown means, as part of its' adrenergic mandate. Aside from TH facilitating a lowering of PDE activity, TH has other beneficial metabolic targets in PE, described below. Thus it was hypothesized that treatment with T3 will ameliorate the maternal PE syndrome pathophysiology 13.

Genomic imprinting {9} is an epigenetic phenomenon that causes genes to be expressed in a parent-of-origin-specific manner. It is estimated that there are approximately 80-100 imprinted genes known to exist in humans. Many of these human imprinted genes are involved in embryonic and placental growth and development. Around 80% of imprinted genes are found in clusters, known as imprinted domains, suggesting a level of coordinated control. The grouping of imprinted genes within clusters allows them to share common regulatory elements, such as non-coding RNAs and differentially methylated regions. When these regulatory elements control the imprinting of one or more genes, they are known as imprinting control regions (ICRs). A widely accepted hypothesis for the evolution of genomic imprinting is the ‘parental conflict hypothesis’. This hypothesis states that the inequality between parental genomes due to imprinting is a result of the differing interests of each parent in terms of the evolutionary fitness of their genes. Genomic imprinting has been found in all placental mammals, where post-fertilization offspring resource consumption, at the expense of the mother, is high.

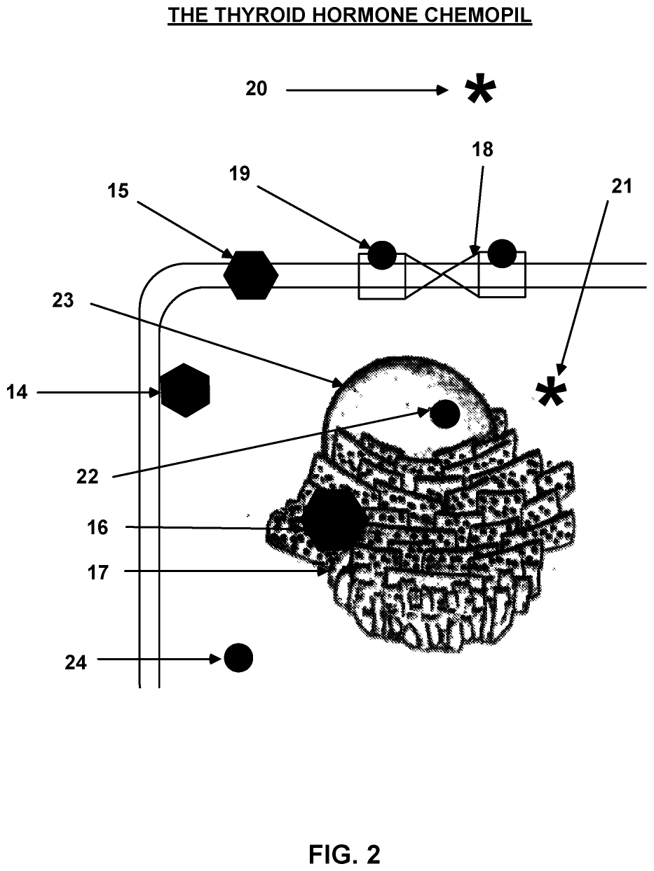

FIG. 2 is a diagram of the THC. DI type 1 (D1) 14 is located in the cytoplasm at the periphery of the cell. DI type 3 (D3) is located in the plasma membrane of the cell 15. DI type (D2) 16 is located in the ER 17. Monocarboxylate transporter 8 (MCT8) 18 is an example of one of a number of TH membrane transporters located in the plasma membrane in association with the TH receptor 19. An extracellular signaling molecule 20 is noted, as is an intracellular signaling molecule 21. A nuclear TH receptor 22 is found inside the nucleus 23. A cytoplasmic TH receptor 24 is noted. Until now, the THC has only been recognized in terms of its' separate components. For the purpose of understanding the present disclosure, the THC is concretized into a stand-alone entity, for it is this entity against which the PIG wages its' attack on the mother. The THC is a complex relay system modulating TH agonist activity in virtually every cell in the body by the use of extracellular and intracellular components and signaling by chemical messengers. The signals are transmitted and/or received by the DI enzymes. Those elements other than DI enzymes sending or receiving these signals are presently not well understood but would likely include TH receptors (membrane, cytoplasmic and nuclear) as well as TH membrane transporters. The THC regulates the hypothalamic-pituitary-thyroid axis (HPTA) as well as TH actions in the periphery. With the advent of new knowledge regarding the DI enzymes and related signaling molecules, it has been recognized that actions of the THC in the periphery may occur independently of the HPTA axis {1}. What this means is that the thyroid function tests, traditionally regarded as the gold standard of thyroid function, may not correlate with TH function in the periphery. With the existing paradigm of thyroid hormone kinetics and dynamics, a normally functioning HPTA (by whatever means this is determined) guarantees normal TH dynamics in the periphery. The recognition that the THC has actions on the peripheral tissues distinct from the HPTA, and without input from the HPTA, introduces a paradigm shift in the understanding of TH dynamics. This concept has not yet gained wide recognition.

The components of the THC include, but are not limited to:

1. The DI enzymes.

2. TH receptors.

3. TH membrane transporters.

4. Signal generators which are not DI enzymes or receptors.

5. Signal recipients which are not DI enzymes or receptors.

6. A family of signaling molecules.

The THC regulation of the BoTH is critical both in ante-natal and in post-natal life:
1. The placenta must protect the developing fetus from the unwanted effects of maternal TH. In the developing fetus, when TH is in the wrong place at the wrong time, TH is teratogenic. The feto-placental unit uses two mechanisms to make certain that TH activity in the developing fetus occurs only where and when it is needed {3}: (i): Placental D3 activity is robust, inactivating maternal TH in order to minimize passage to the fetus; (ii): The availability of TH in fetal tissue is tightly regulated in space and time. At a place and time where TH agonist activity is needed, D2 is upregulated and D3 is downregulated. When the need for TH activity no longer exists, the reverse occurs with D2 downregulated and D3 upregulated. This micromanagement of DI activity in space and time is one of the most critical fine-tuning modalities of embryogenesis. Without the THC playing this role in embryogenesis, the human embryo could never develop normally.
2. Following birth the THC shifts to a post-gestational mode, which is characterized by ultra-fine tuning of TH activation by D2 and near dormancy of D3 in all tissues with the exception of the brain and skin. This persistence of post-gestational D3 activity in the brain has negative implications in the patho-physiology of Alzheimer's disease.

The recognition of the THC and its independence from the HPTA opens the door for the recognition of a panoply of conditions, heretofore known as Thyroid Hormone Dysregulation Syndromes (THDS) which encompass all aberrations of thyroid hormone dynamics, many of which are not presently recognized as being associated with aberrations in TH dynamics, and most of which occur in the presence of a normal plasma TSH level and a normal plasma free T4 level.

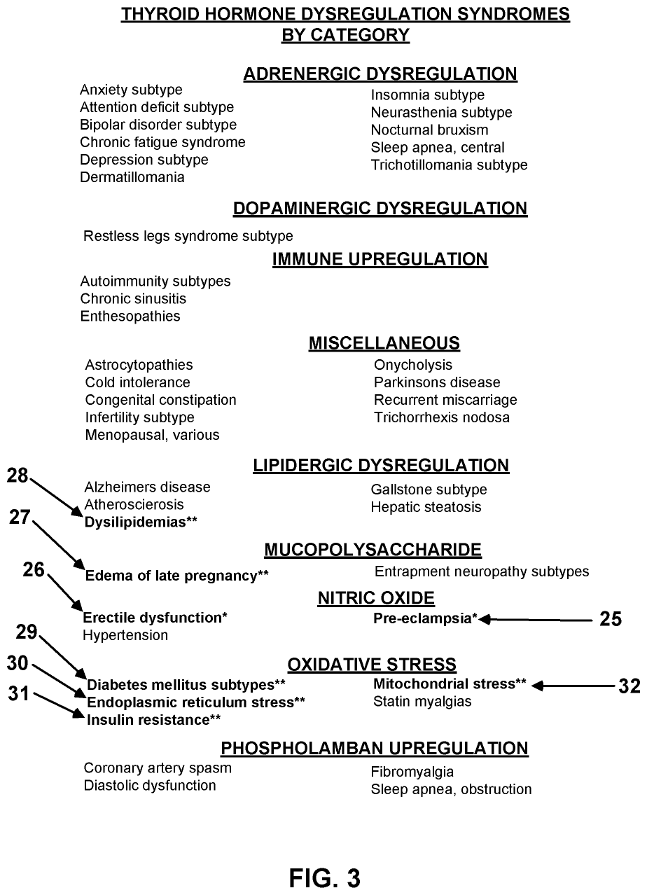

FIG. 3 is a list of over 40 such Thyroid Hormone Dysregulation Syndromes. Only a few are highlighted because of relevance to the present disclosure. Pre-eclampsia 25 and erectile dysfunction 26 are presented in bold type, single asterisk, as they have relevance to the present disclosure, inasmuch as each is a THDS with a NO deficit which responds to PDE inhibition. Also in bold type, double asterisk, are edema of late pregnancy 27, dyslipidemias 28, diabetes mellitus subtypes 29, endoplasmic reticulum stress 30, insulin resistance 31, and mitochondrial stress 32, all co-morbidities of PE. The listing of a condition in FIG. 3 does not necessarily imply that the condition can simply be cured by T3 administration. The issues are far more complex. Rather, there is the implication that TH dysregulation is involved in some way in the pathophysiology, in a manner suggested by the sub-category in which the condition is listed.

FIG. 4 is a diagrammatic representation of the PIG attack on the THC of the mother. The upper portion of the diagram shows the feto-placental unit 33. The lower portion of the diagram shows the mother 34. The PIG 35 complex is shown. The PIG 35 complex orchestrates a hijacking of placental metabolism 36 and uses it to develop an attack weapon 37. The attack weapon takes the form of upregulation of sonic hedgehog signaling 38 {10} and upregulation of transformational growth factor beta (TGF-beta) 39 activity. The significance of these changes will be explained below. These are the two key elements of the attack on the mothers THC 40. Concentrations of both the sonic hedgehog signaling molecule {10} and TGF-beta {11} have been found to be elevated in the blood of pregnant women with PE. Sonic hedgehog is known to ubiquitinate D2 {1}, reversibly inactivating it and decreasing the capacity of D2 to activate TH. TGF-beta is known to upregulate D3 {12}, thereby increasing the inactivation of T4 by inner ring deiodination. The significance of these aberrations of D2 and D3 must be explained further in order to reinforce their significance. The teleologic significance of the THC's priority to prevent unwanted TH activation has been referenced above. There is perhaps no more dramatic evidence for this than the fact that the chief inactivator of TH, D3, has a half-life of 12 hours, while the chief activator of TH, D2, has a half-life of 40 minutes. D2 is a delicate and vulnerable enzyme primarily because of its short half-life, which can be further shortened under certain conditions. Gereben has noted {1} that ‘D2 is considered the critical homeostatic T₃-generating deiodinase due to its substantial physiological plasticity. A number of transcriptional and posttranscriptional mechanisms have evolved to ensure limited expression and tight control of D2 protein levels, which is critical for its homeostatic function. D2 activity/mRNA ratios are variable, indicating that there is significant posttranslational regulation of D2 expression. In fact, the decisive biochemical property that characterizes the homeostatic behavior of D2 is its short half-life (˜40 min), which can be further reduced by exposure to physiological concentrations of its substrate, T₄, and in experimental situations, rT₃ or even high concentrations of T₃. This down-regulation of D2 activity by substrate is a rapid and potent regulatory feedback loop that efficiently controls T₃ production and intracellular T₃ concentration based on availability of T₄ ’ Thus D2 is posttranslationally downregulated by sonic hedgehog {10}, reducing the activation of TH; and D3 is upregulated by transcriptional stimulation mediated by Smad (the family of signaling molecules associated with TGF-beta activity) signaling {12}. The effect of sonic hedgehog on D2 and the effect of TGF-beta on D3 can be applied in an attempt to explain the thyroid function tests noted to be associated with PE. It has been consistently found in cohorts of patients with PE that, compared with controls, TSH levels are mildly elevated and free T4 and free T3 levels are lower than controls {13}. Sonic hedgehog inhibition of D2 may explain the lower T3 levels while the lower levels of T4 may be explained by increased breakdown of T4 by D3 upregulated by TGF-beta. In order for TSH suppression to occur, T4 must be presented to the tissues of the hypothalamus and pituitary where it enters the cells with the thyrostat function in which it is then deiodinated by D2. The TSH elevations in PE may be explained by the presentation of less T4 to the thyrostat tissues and the inhibition of D2 by sonic hedgehog. While D2 present in the hypothalamus and pituitary is known to be more resistant to ubiquitination compared with D2 in the periphery, there is no evidence that it is immune to being ubiquitinated.

Thus the patient with PE develops a serious disturbance in the BoTH. Consequently, the metabolism of the mother suffers a unique form of cellular hypothyroidism, lacking in many of the classical signs, one whose clinical signs are masked by the pregnant state. Whether the edema fluid in PE constitutes myxedema is a question that appears not to have been addressed. It has been assumed that the peripheral edema seen in PE is ‘pregnancy edema’, only worse. Maternal skin fibroblasts in PE are known to produce excess amounts of certain mucopolysaccharides. This is a hallmark of hypothyroidism. Studies of the constituents of PE edema fluid are believed not to have been published in the past 40 years. Those published prior did not analyze for mucopolysaccharides.

One of the chief consequences of this ‘masked hypothyroidism’ is a failure to sustain the generation of a sufficiency of the gasotransmitters NO, CO. This arises because the subthreshold thyroid hormone activity is insufficient to maintain normal activity of enzymes NOS and HO and is also insufficient to maintain adequate suppression of the PDE enzyme activity, as will be discussed in detail below. The damage inflicted by the PIG gene to the maternal metabolism is permanent, conferring a lifelong increased risk of cardiovascular disease (coronary artery disease, hypertension and stroke) and syndromes of insulin resistance (metabolic syndrome, type 2 diabetes mellitus). Because of its' association with ER stress, insulin resistance and aberration in the BoTH, Alzheimers disease may be an additional late consequence of PE, although this has not been studied. The mechanisms by which this lifelong risk of disease comorbidities is inflicted on the mother are unknown. If they are primarily related to permanent aberration in the maternal THC, the details have still to be elucidated. It is proposed here that PE is caused by a rogue PIG or a cluster of PIGs constituting an Imprinted Domain, and controlled by an Imprinting Control Region. The motivation of the PIG is to optimize success of the fetus at the expense of the mother. The PIG is ruthless in its' quest for this goal. The precise outcome cannot be guaranteed, but the PIG is prepared to roll the dice.

Outcomes include:

1. Death of mother and fetus.

2. Death of mother with survival of the fetus.

3. Mother survives, with permanent damage to her metabolic infrastructure, and the fetus dies in utero or following birth.

4. Neither mother nor fetus die, but the mothers' reproductive health is compromised by aberrations in TH production and/or dynamics which lead to infertility or recurrent miscarriage.

In the case of preservation of the life of the child, the goal of the PIG is to limit competition from siblings, giving the fetus a post-natal advantage.

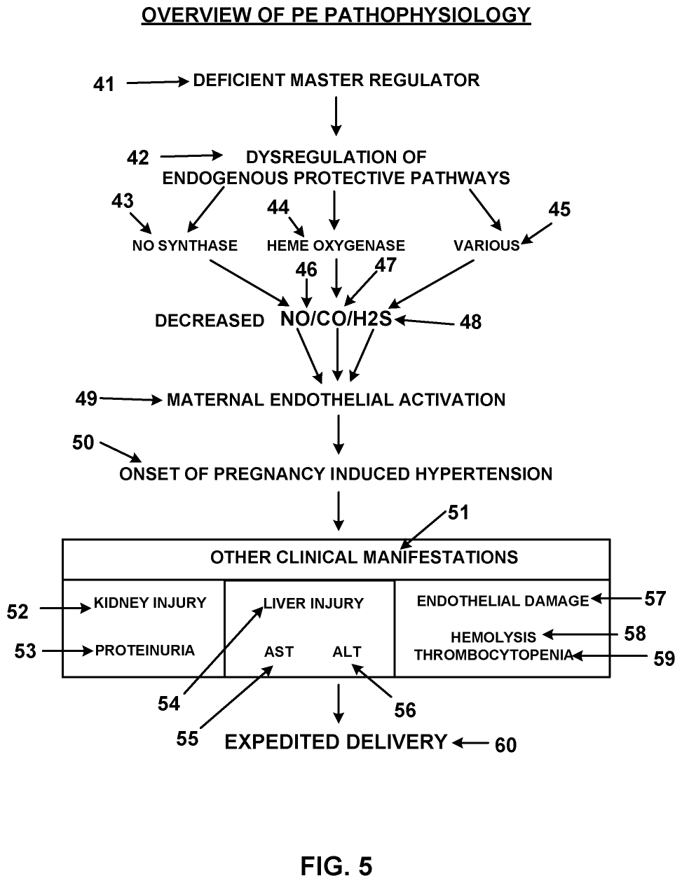

FIG. 5 shows an overview of PE pathophysiology. In the face of the deficient master regulator 41 (proposed in the present disclosure to be T3), dysregulation of endogenous protective pathways 42 occurs. NOS 43 activity is reduced and HO 44 activity is reduced. Various 45 enzymes responsible for generating H2S 48 (referenced above) are dysregulated. Consequently the production of NO 46, CO 47 and H2S 48 is reduced. This results in maternal endothelial activation 49 and the onset of hypertension 50 associated with PE. Aside from hypertension 50 the other clinical manifestations are kidney injury 52 with proteinuria 53, liver injury 54 with elevated liver enzymes aspartate amidotransferase 55 and alanine amidotransferase 56, and endothelial damage 57 manifesting as hemolysis 58 and thrombocytopenia 59. The maternal PE syndrome frequently reaches a critical crescendo prior to term and emergent expedited delivery 60 must be achieved, often by cesarean section.

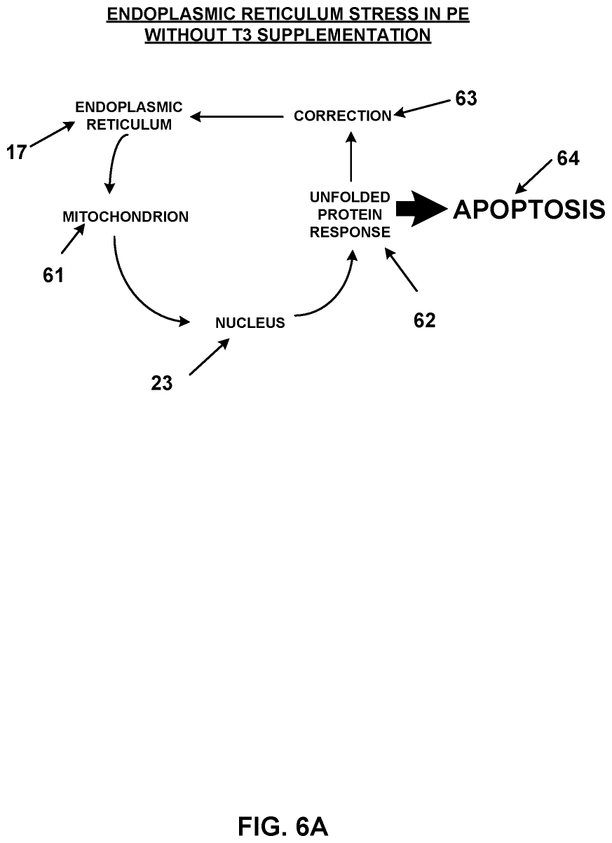

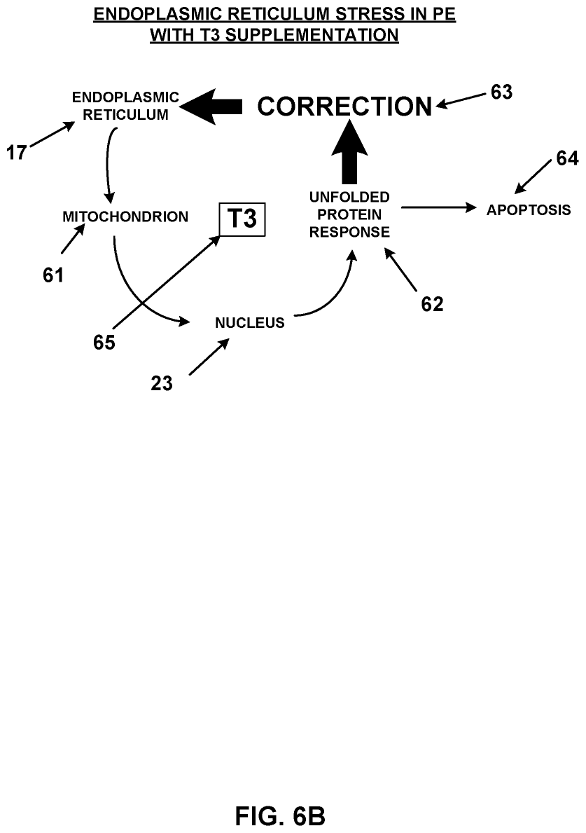

FIG. 6A shows ER stress as a manifestation of the oxidative stress in PE. The ER 17 is shown in the schematic together with the mitochondrion 61 and the nucleus 23. ER stress is to be found in numerous conditions, including Alzheimers disease and Type 2 diabetes mellitus. It is caused by cellular hypothyroidism, the Thr92Ala polymorphism of D2, as well as conditions unrelated to TH dynamics. Regardless of the cause of ER stress, the molecular biologic derangements are complex. One of the most important of these derangements is disruption of the activity of D2 which is resident in the ER {2}. The disruption of D2 activity eliminates a key remedy for the existing oxidative stress and, at the same time, worsens the oxidative stress further. The reason for this is that T3 is the most potent known physiologic regulator of mitochondrial function, both qualitatively and quantitatively. Robust mitochondrial function is necessary in order for the cell to generate sufficient redox capacity to prevent or counteract the oxidative stress. The unfolded protein response (UPR) 62 is activated in the ER by the oxidative stress. The UPR activation occurs in response to an accumulation of unfolded or misfolded proteins in the ER lumen. The UPR aims to restore normal function to the cell by halting protein translation, degrading misfolded proteins and activating signaling pathways directed to increasing the production of molecular chaperones involved in protein folding. If these initial UPR goals are not met within a certain time frame, the UPR shifts its goal to achieving apoptosis, the death of the cell. Under these circumstances of cellular hypothyroidism, the UPR lacks the resources to correct the situation. Correction 63 in the ER is minimal and apoptosis 64 is the main outcome.

FIG. 6B shows ER stress in PE when T3 65 is administered to compensate for the deficient D2 activity in the ER. The schematic shows the ER 17, the mitochondrion 61 and the nucleus 23. The supplemented T3 65 is now able to fulfill its' molecular biologic mandate. Qualitative and quantitative mitochondrial function is improved, increasing redox capacity and ameliorating the oxidative stress. The UPR 62 triggered is now able to maximize the required correction 63 in the ER and to minimize apoptosis 64. The benefits of T3 administration expanded upon here may be seen in other conditions characterized by ER stress and/or oxidative stress including, but not limited to, type 2 diabetes mellitus and Alzheimer's disease. The critical role of T3 here, literally saving the life of the cell, is the restoration of the redox balance in the cell. This occurs via an increase in qualitative and quantitative mitochondrial function and an upregulation of the enzymes involved in glutathione synthesis, resulting in a substantial increase in the number of free radical scavengers.

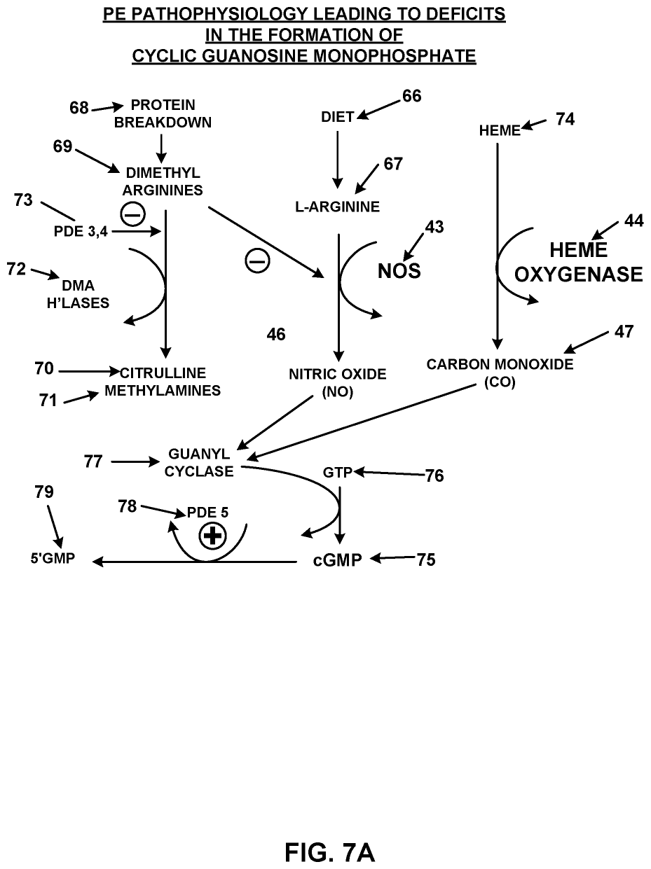

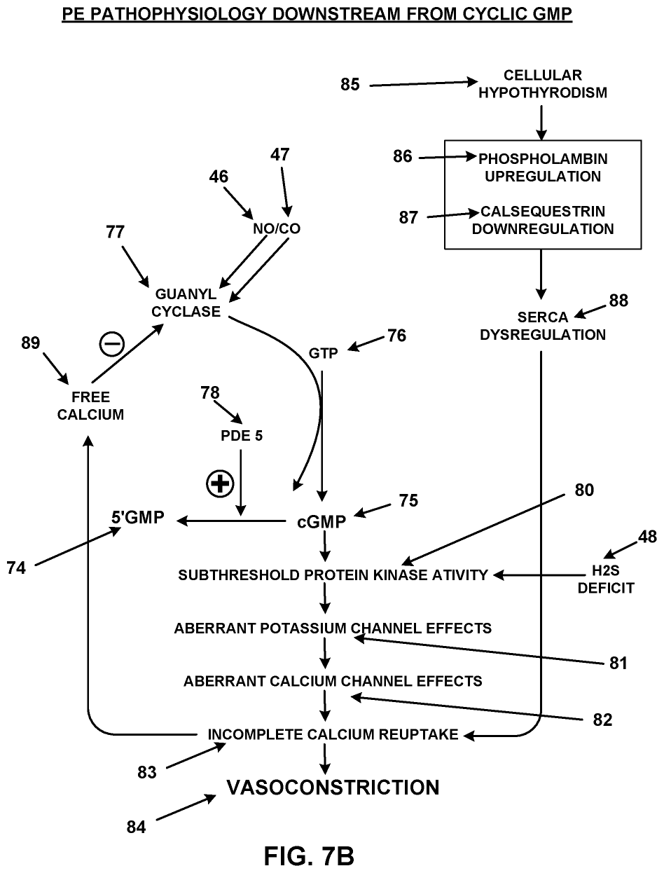

FIG. 7A shows the deficits in the generation of the gasotransmitters NO 46 and CO 47 in PE leading to subthreshold production of cGMP 75, with downstream consequences shown in FIG. 7B. Diet 66 is the chief source of L-arginine 67, the substrate in the NOS 43 reaction which donates the nitrogen atom to form the molecule of NO 46 generated. Protein breakdown 68 yields assymetric dimethylarginines (ADMAs) 69 which competitively inhibit the NOS 43 reaction {13}. The ADMAs 69 are catabolized to citrulline 70 and methylamines 71 by the group of enzymes known as dimethylarginine hydrolases 72. The dimethylarginine hydrolase 72 reactions are slowed by PDE 3/4 73 activity. Heme 74 is the substrate for the HO 44 reaction whereby a molecule of CO 47 is generated. ADMAs 69 are increased in pregnancies complicated by PE {14}. L-arginine 67 levels are reduced in pregnancy {15}. Dietary L-arginine 67 supplements have been shown to significantly reduce, but not eliminate, the incidence of PE in high risk patients {15}. In the setting of a surfeit of T3 65 and in the absence of the pharmacologic supplementation of the present disclosure, the following aberrations to gasotransmitter generation occur.
(i) Subthreshold TH reduces the efficiency of the NOS 43 reaction.
(ii) Subthreshold TH reduces efficiency of the HO 44 reaction.
(iii) The dimethylarginine hydrolase 72 reactions are slow, resulting in the buildup of significant concentrations of dimethyarginines 74 which competitively inhibit the NOS 43 reaction.

The net effect of these aberrations is reduced production of NO 44 and CO 45 which reduce the efficiency of guanyl cyclase 77. This reduces the amount of cGMP 75 produced. Cyclic GMP 75 is the key intermediary second messenger here translating the sufficiency of gasotransmitter effect into the sequence of downstream effects. Cyclic GMP 75 is formed by the action of the enzyme guanyl cyclase 77. Guanyl cyclase 77 converts guanosine monophosphate 76 to cGMP 75 and requires a sufficiency of NO 46 and CO 47 as cofactors in order to do this. Cyclic GMP is broken down to 5′GMP 79 by PDE 5 78.

FIG. 7B shows PE pathophysiology downstream from cGMP. FIG. 7B again shows the synthesis of cGMP 75 from GTP 76 facilitated by guanyl cyclase 77 in the presence of NO 46 and CO 47, and its' breakdown to 5′GMP 79 by PDE 5 78. In PE, and given the upstream pathophysiology described in FIG. 7A, subthreshold amounts of cGMP 75 are produced. This results in subthreshold protein kinase 80 activity, a phenomenon aggravated by a surfeit of H2S 48. Aberrant potassium channel 81 effects result, followed by aberrant calcium channel 82 effects leading to incomplete calcium reuptake 83 from the cytosol into the sarcoplasmic reticulum (SR), and consequent vasoconstriction 84. The euthyroid state facilitates complete reuptake of calcium into the SR. SERCA 88 is the pump mechanism which performs this function with the assistance of calsequestrin 87, whose function is to maintain a low concentration of free calcium in the SR so that the gradient against which SERCA 88 must pump is kept low. Phospholamban 86 is a protein which inhibits SERCA 88, making it unable to function. A sufficiency of TH is accompanied by upregulation of the calsequestrin 87 gene {16}. TH also downregulates the phospholamban 88 gene {17}. In the presence of cellular hypothyroidism 85 the calsequestrin 87 gene downregulates. and the phospholamban 86 gene upregulates. There is increased transcription of phospholamban 86 which is in the unphosphorylated state. In this form phospholamban 86 inhibits SERCA 88. To make matters worse, the level of free calcium 89 present in the cytosol (which should be negligible) results in downregulation of guanyl cyclase 77 {18} in a dangerous repeating cycle which results in feedback inhibition of cGMP production. This feedback inhibition of cGMP compounds the physiologic trespass from the upstream gasotransmitter aberrations.

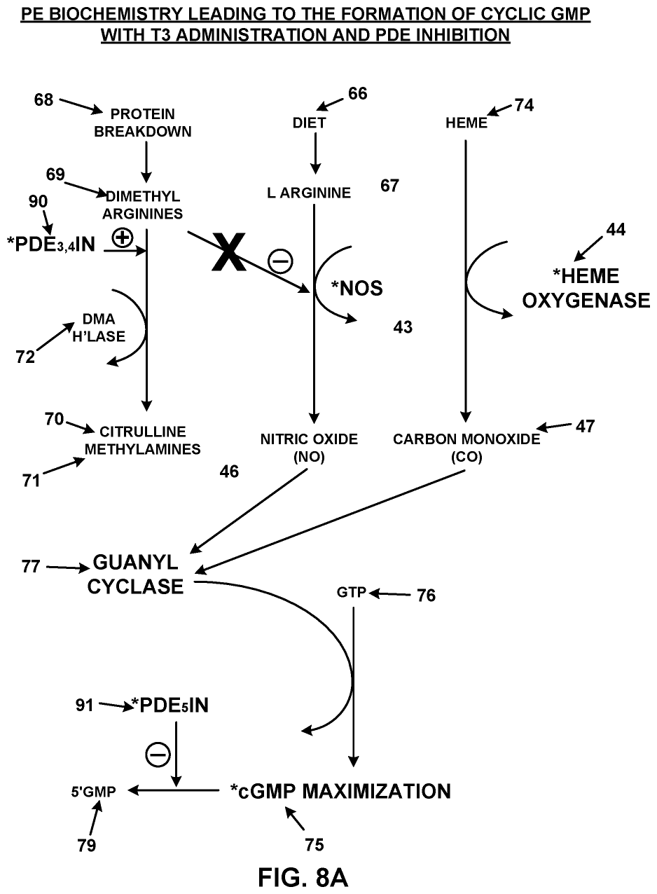

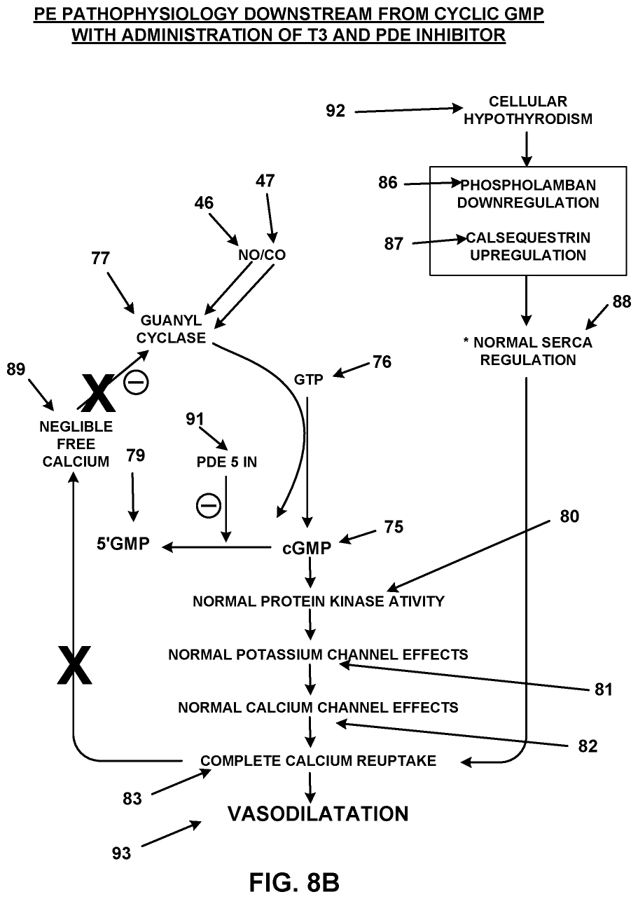

FIG. 8A shows the corrective effects on the pathophysiology of FIG. 7A when T3 65 or T3 65 plus the stated classes of PDE inhibitors are administered to a patient with incipient or actual PE. L-arginine 67 sourced from the diet 66 donates the nitrogen atom which generates NO 46 with the catalytic help of NOS 43. This reaction is facilitated by a sufficiency of T3 65 which upregulates NOS 43 {19}. HO 44 catalyses the generation of a molecule of CO 47 from heme 74. This reaction is also facilitated by a sufficiency of T3 65 {20} which upregulates HO 44. ADMAs 69 are products of protein breakdown. They are catabolized by dimethylarginine hydrolases 72, broken down to citrulline 70 and methylamines 71. The reactions are slowed in the presence of PDE 3/4 73 activity. Administration of a PDE 3/4 inhibitor 90 increases the efficiency of the dimethylarginine hydrolases thereby reducing the concentrations of ADMAs 69. This intervention has been shown to increase NO production in certain forms of pulmonary hypertension where similar NOS pathophysiology exists {21}. This is important because these ADMAs competitively inhibit the NOS 43 reaction. Thus it is shown that the administration of a PDE 3/4 inhibitor 90 reduces or eliminates the inhibition of the ADMAs 69 on the NOS 43 reaction. Cyclic GMP 75 is a critical chemical moiety in the pathophysiology of PE. Factors determining the production of cGMP 75 are in delicate balance. In the presence of compromised guanyl cyclase, cGMP 75 levels are suboptimal and what cGMP there is, is broken down to guanosine monosphate (5′GMP) 76 by PDE 5 78, compromising the chain for the maintenance of downstream vasodilatation. The use of a PDE 5 inhibitor 91 has been shown to have salutary effects in animal PE studies {22}, males with ED and in certain forms of pulmonary hypertension. All three of these conditions are known to be associated with similar, if not identical, NOS/NO aberrations. Thus it is seen from FIG. 8A that four loci of corrective intervention of the pathophysiology of PE, each marked with and asterisk, are highlighted: Upregulation by T3 65 of NOS 43 and HO 44 constitute two of the four. PDE 3/4 inhibition 90 is the third and PDE 5 inhibition 91 is the fourth. These interventions maximize NO 46 and CO 47 production and, collectively, maximize the activity of guanyl cyclase 77 which maximizes production of cGMP 75, the downstream benefits of which will be demonstrated in FIG. 8B.

FIG. 8B shows the corrected effects on pathophysiology of FIG. 7B downstream from cGMP when T3 65 and PDE inhibitors 90, 91 are administered to a patient with incipient or actual PE. Guanyl cyclase 77 activity is upregulated and optimized. It is optimized by a sufficiency of the gasotransmitters NO 46 and CO 47. cGMP is upregulated because there is now negligible free calcium 89 in the cytosol which, if present, would downregulate it. This is explained below. Cyclic GMP 75 levels are maximized by the robust activity of guanyl cyclase 77 and by inhibition of the breakdown of cGMP 75 to 5′GMP 79 by the PDE 5 inhibitor 91. The maximization of cGMP 75 facilitates normal protein kinase 80 activity. This results in normal potassium channel 81 effects which lead to normal calcium channel 82 effects. The normal calcium channel 82 effects, coupled with the normalized SERCA 88 function (explained below) result in complete calcium reuptake 83 from the cytosol into the SR, thus facilitating vasodilatation 93. The T3 65 administration restores cellular euthyroidism 92. This normalizes SERCA 88 (marked with an asterisk) regulation by downregulating phospholamban 86 and upregulating calsequestrin 87. Thus this TH action normalizing SERCA function constitutes the fifth locus of efficacy of the present disclosure, the first four having been referenced in FIG. 8A. Thus there are five salutary loci of inflection of the present diclosure on maternal PE pathophysiology. Sildenafil (a PDE 5 inhibitor) at one time showed promise as a stand-alone therapy for PE. Given that sildenafil only corrects one inflection locus (cGMP 75 to 5′GMP 79), it is not surprising that sildenafil monotherapy has been an incomplete success and that the drug has not been adopted as a treatment for women with PE. H2S 48 is not referenced in FIG. 8B as salutary effects of the present disclosure on H2S generation are, as yet, not shown to exist and the role of H2S in PE has yet to be clearly defined. The current understanding of the effect of TH on H2S production is that TH suppresses H2S production by downregulating two or more of the four enzymes responsible for H2S synthesis. Conflicting information exists on the role of H2S as an independent variable in PE pathophysiology and at the time of writing there is no evidence to suggest that boosting H2S production alone reverses maternal PE pathophysiology. In fact, there is some evidence that H2S has functions which differ from those of NO and CO. CBS, one of the enzymes responsible for H2S synthesis, has binding sites for NO and CO. When these sites are occupied by NO and CO and the CBS-associated Fe ion is in the ferrous state, the enzyme is inhibited. This suggests that under certain circumstances H2S actions are opposed to those of NO and CO. As a consequence it must be stated that no claim is made in the present disclosure in regard to TH increasing H2S levels in PE.

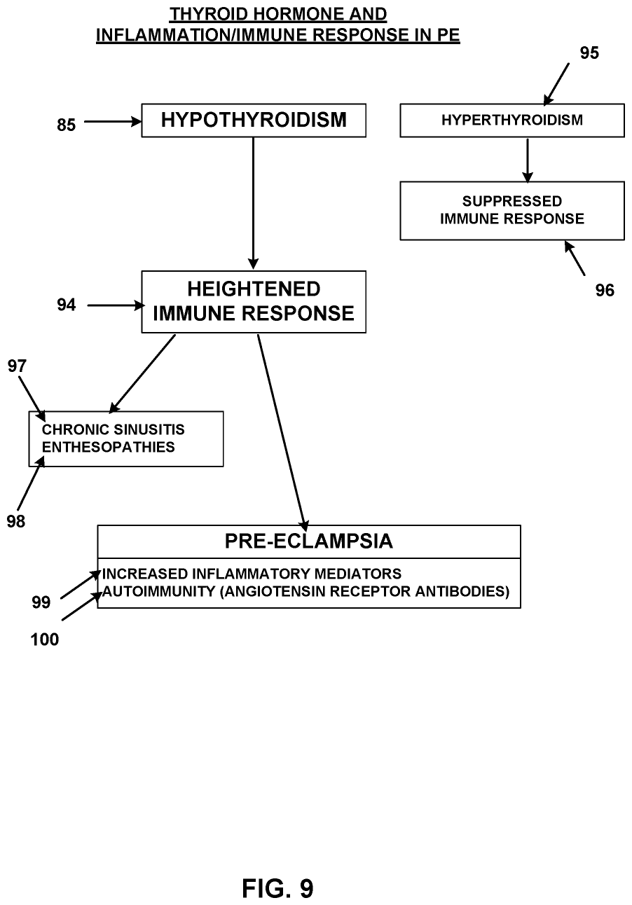

FIG. 9 concerns extreme TH states as they relate to inflammation and the immune response. Hypothyroidism 85 is generally considered to be associated with an exaggerated immune response 94 while hyperthyroidism 95 is associated with a moderately suppressed immune response 96. In certain non-pregnant hypothyroid patients evidence of a ramped up immune response reflecting cellular hypothyroidism may be seen in the form of chronic sinusitis 97 or painful enthesopathies 98. Although a cause effect relationship in PE is not proven, it is certainly plausible that cellular hypothyroidism 85 in PE accounts for at least some of the inflammation, evidenced by increased inflammatory mediators 99 seen in PE as well as the autoimmunity involving such targets as the angiotensin receptor AT1 100.

Once the art of the present disclosure has undergone derivative research, this research will enable new art to be developed relating to early diagnosis of PE. Said art will involve genetic testing applications of maternal peripheral blood sampling, chorionic villus sampling, umbilical cord blood sampling, amniocentesis as well as the pre-existing art of exhaled NO sampling. These techniques will be used to identify pregnant patients at risk for development of PE prior to the development of proteinuria and/or hypertension. The referenced modalities will be used to identify the PIG gene or downstream effects of the PIG gene.

A subset of patients taking T3 monotherapy (T3 without T4) will show thyroid function tests which demonstrate an apparently spurious rise in TSH. This occurs because the level of plasma T3 generated in these patients is insufficient to induce central negative feedbac inhibition/suppression of TSH. This central negative feedback inhibition/suppression of TSH is primarily a T4 mediated phenomenon, mediated by T3 only at higher blood levels in certain patients. The origin of the apparently spurious rise in TSH is explained here. While the therapeutic T3 level in this subset of patients is too low for central negative feedback inhibition/suppression of TSH, it is not too low to produce negative feedback directly to the thyroid gland. This effect reduces production and secretion of T4 by the thyroid gland. As a consequence, the plasma level of T4 falls, reducing the central feedback inhibition/suppression of T4 on the central apparatus and thus the TSH rises. This phenomenon results in an elevated TSH, suggesting a hypothyroid state, when in fact the patient is euthyroid by virtue of the T3 treatment. Another mechanism by which the TSH might rise with T3 monotherapy is the induction of ubiquitination in the central thyrostatic cells by elevated T3 levels. Because of the relative resistance of D2, located centrally, to ubiquitination, this should only occur with intermediate or high doses of T3.

Therefore, in another embodiment, ERT3 may be formulated together with T4, or the two may be given in separate formulations at the same time, thereby maintaining T4 levels with a sufficiency such that central negative feedback inhibition is maintained and a normal TSH is preserved. It should be noted that, because of the low potency of T4 (it is the pre-hormone) and because of its' relatively long half-life of 5-7 days, there is no advantage to be gained by providing T4 in the formulation of the present disclosure in an extended release format. Whether the T4 formulated for the present disclosure is immediate release or extended release is merely a matter of convenience for the pharmacist. Thus the formulation of T4 in the present disclosure allows for either immediate release or extended release T4. Any new art coming after the fact and attempting to circumvent the intellectual property derived from the present disclosure by introducing claims language for the addition of extended release T4 to the formulation should be considered specious.

Extended release/controlled release/delayed release dosage forms have been used since the 1960s to enhance performance and increase patient compliance while also potentially minimizing unwanted side effects. The dosage forms may comprise those configured to release the active ingredient over a four-hour period, or over an eight-hour period, or a twelve, twenty-four hour, thirty-six hour, or even forty-eight hour period. Alternately the release may be a delayed release in that the active ingredient doesn't reach significant levels in the blood until about one to four hours after dosing with release over the next twenty-four to twenty-six hours or more including over thirty-six or forty-eight hours. In regard to the present disclosure, total twenty-four hour or daily intake of the active ingredient, L-tri-iodothyroinine, may be at least 1 μg, or 2 μg of active ingredient, or at least 4 μg, or at least 6 μg, or at least 8 μg, or at least 10 μg, or at least 12 μg, or at least 14 μg, or at least 16 μg. In other embodiments, the unit dosage form may comprise one or more extended-release dosage forms which are configured to release the active ingredient over a period of days such as in the case of the internal implanted device. Oral extended release or controlled release formulations may be of several types. Matrix type extended release systems or diffusion-controlling membranes, or other extended release technologies may be employed. Non-active inert ingredients, which may also be excipients for drug delivery and/or needed for formulation may be included. In another embodiment, ERT3 may be formulated together with T4, or levothyroxine or L-thyroxine, to maintain TSH levels in the normal range, as discussed above. Levothyroxine is a synthetic thyroid hormone that may be available under the names Levothroid, Levovyxl, Levo-T, Synthroid, Tirosint, and Unithroid. Thus, it is appreciated that the optimum pharmaceutical in the instant case may be an extended release formulation of T3, with T4 added, and with variable T4/T3 ratios allowing for customized patient formulation. Multiple dosage permutations, including differing ratios of T3 to T4, are another objective of the present disclosure. In the embodiment combining T3 and T4, the total daily dose of T4 may be 20, 40 or 60 mcg, or a different dose. The total daily dose of T3 may be between 2 mcg and 18 mcg, or a different dose. Thus with the foregoing schedule (T4=20, 40 or 60; T3=2-18), the 12 hourly dose would be as low as T4:T3=10:1 and as high as 30:9, with all possible permutations in between. It is anticipated that the optimum dosing interval will be every 12 hours, although other intervals may also be appropriate without departing from the spirit and scope of the invention.

The half-life of T3 in humans is 19 hours {23}. With dosing of ERT3 every 12 hours, low steady state plasma levels of T3 will be attained while ensuring continuous genomic and non-genomic effects, without the adverse effects of immediate-release T3. The method may result in blood levels of T3 more closely approaching steady state blood levels compared with the administration of an immediate release formulation of T3.

Further, the method of the present disclosure may be used in patients with TSH levels which are within the normal ranges. The method may also be used in patients with TSH levels which are outside the normal ranges. Because T3 is a stimulating hormone, excess can lead to cardiac complications which include cardiac hypertrophy, arrhythmias and high output heart failure. Even in the absence of sustained chronic T3 excess, immediate release T3, with its' supraphysiologic post-absorptive plasma levels, may produce cardiac arrhythmias, chiefly supraventricular. Therefore, immediate release T3 is not suitable. Absorption of T3 (L-triidothryonine or liothyronine) is 90% with peak levels reached one to two hours following ingestion. Serum concentration, or amount of drug in circulation, may rise by 250% to 600%. Single dose, immediate release T3 ingestion may place a patient at risk for cardiac arrhythmias, chiefly but not limited to supra-ventricular arrhythmias, and potentially other adverse effects.

A method for treating PE with T3 being L-triiodothyronine, liothyronine, liothyronine sodium, or similar formulations in an extended release system allows patients to be treated for PE in a safe manner. Extended release caplets or tablets or other suitable vehicle for administration, being via oral, injectable, or other suitable route of administration to a human patient, not limited to a tablet, capsule, gelcap, a powder dispensed in a beverage, orally disintegrating tablet, a vial, ampule, or other container of liquid such as a solution or suspension, a lozenge, lollipop, gum, inhalers, aerosols, injectables, creams, gels, lotions, ointments, balms, eye drops, suppostitories, and patches , with the minimum T3 dose, tailored to the individual patient for body weight and age overcomes these concerns resulting in lower blood levels. Alternately a drug dispensing device may be implanted either sub-dermally or otherwise and configured to release T3 in a slow manner. The post absorptive blood levels of this extended release T3 could more closely resemble a steady state or constant level of T3 in the blood rather than a high spike in post-absorptive blood levels of the immediate release formulation, thereby avoiding supra-physiologic or high serum concentration of T3 levels in the blood. Matrix type extended release systems or diffusion-controlling membranes, or other extended release technologies may be employed. Non-active inert ingredients for drug delivery may be included in formulations. Matrix type systems may be based on hydrophilic polymers wherein the drugs and excipients, being non-active inert ingredients, are mixed with polymer such as hydroxypropyl methylcellulose (HPMC) and hydroxypropyl cellulose (HPC) and then formed as a tablet by conventional compression. Water diffuses into the tablet, swells the polymer and dissolves the drug or active ingredient, whereupon the drug may diffuse out being released into the body. This type of controlled or extended release technology is open to mechanical stress from food substances which may lead to increased release rate and a higher risk of dose-dumping. These systems also require a large amount of excipient and drug loading is comparatively low. Diffusion-controlling membranes is another method of obtaining extended or controlled release of active ingredients. With this technology, a core that may be pure active ingredient, or mixture of active ingredient and excipient(s), is coated with a permeable polymeric membrane. Water diffuses through the membrane and dissolves the drug which then diffuses out through the membrane at a rate determined by the porosity and thickness of the membrane. Membrane polymers may be those such as ethylcellulose.

Attention now turns to defining embodiments of the present disclosure; the composition of the pharmaceutical and the optimum manner in which it is administered to patients in order to prevent or treat the maternal PE syndrome. Two example formulations, with example dosing, of the present disclosure are shown:
1: ERT3 (with or without T4) with no PDE inhibitors.
2: ERT3 (with or without T4) combined with specified PDE inhibitors.

In regard to example 1, and for reasons discussed, immediate release T3 is not suitable. The form of T3 used is ERT3, with T4 optionally added for maintaining the TSH level in the normal range. It is expected that the 24 hour dose of T3 required for prevention or treatment of pre-eclampsia will be between 2 and 18 mcg. The formulation will be an extended release formulation of T3, with T4 optionally added, in dosages as specified hereunder. The optimum dosing interval will be every 12 hours but may be an interval that is shorter of longer. Specific dosing of ERT3 with T4 is used according to the present disclosure in order to customize the pharmaceutical to the individual patient. Daily (24 hour) dosing of T4 of 20 mcg or 40 mcg or 60 mcg or of a different dose are used. Daily dosing of T3 of 2 mcg, 4 mcg, 6 mcg, 8 mcg, 10 mcg, 12 mcg, 14 mcg or 16 mcg or 18 mcg of a different dose are used. A majority of patients will require T3 dosing to be 2-18 mcg per 24 hours (with many requiring doses at the lower end of this range), but there will be exceptions. A majority of patients will require T4 dosing to be 20-60 mcg per 24 hours, but there will be exceptions. It is foreseen that multiple dosage permutation will be available in order to customize the pharmaceutical to the needs of the individual patient, by using differing ratios of T3:T4. By means of example, and not limitation or exclusion, a reasonable choice for dosing for a preliminary pilot study would be ERT3, 2 mcg every 12 hours (4 mcg/24 hours) and T4, 10 mcg every 12 hours (20 mcg/24 hours).

In regard to example 2 the same narrative noted above for example 1 regarding the unsuitability of immediate release T3 as well as the narrative regarding ERT3 and T4 administration, as well as expected dosage requirements, applies. Further description of example 2 follows: example 2 is a polypharmaceutical of a combination of drugs active in ameliorating the pathophysiology of PE, namely (i) ERT3 (with or without T4), (ii) a drug that is a combined PDE 3/4 inhibitor (or separate PDE 3 and PDE 4 inhibitors) and (iii) a PDE 5 inhibitor. The advantage of example 2 over example 1 is that example 2 will allow for lower doses of all drugs in the polypharmaceutical (compared with any one drug being used alone) to be effective. This is always of importance when treating a pregnant patient with prescription drugs. Further, it is important that the lowest possible effective dose of ERT3 be used. Example 2 has the most promise for reducing the dosages of all drugs in the polypharmaceutical to levels that are acceptable for use during pregnancy. Work done in rodents has shown that TH modulates cyclic nucleotide PDE activity, reducing it {7} {8}. Thyroidectomized rats demonstrated upregulated PDE activity {7}. In another study rats made hypothyroid demonstrated increased PDE activity which then returned to normal after administration of T3 {8}. The mechanism for this modulation of PDE activity by TH is at present unknown. Thus the downregulation of PDE activity, independently, by TH allows TH to cover all of the five inflection points (marked with an asterisk in FIG. 8A and FIG. 8B) referencing the corrective biochemistry of the present disclosure, without the need for PDE inhibitors. An important purpose of combining a PDE 3/4 inhibitor and a PDE 5 inhibitor with ERT3 is that example 2 will be effective in the management of PE with lower doses of all drugs used compared to the doses required if each drug was used alone. It is anticipated that with example 2, ERT3 doses in the range of 4-8 mcg/24 hours will be effective. It is intuitive that when treating a pregnant patient with a prescription drug, the lowest effective dose should be used. As the present disclosure claims utility of the activity of specified PDE classes, and not specific PDE drugs, whether or not specific PDE drugs are formulated for immediate or extended release will depend on pharmacokinetic factors including, but not limited to, the drug half-life. As an example and in regard to the stated 12 hourly dosing of example 2: with sildenafil, used as the PDE 5 inhibitor (half-life of 4 hours), there may be justification for providing this drug in an extended release format; however with tadalafil, used as the PDE 5 inhibitor (half-life 17-18 hours), extended release formulation would be unnecessary. Thus the PDE inhibitors incorporated into the formulation of the present disclosure may be either immediate release or extended release, depending in the pharmacokinetic characteristics of the specific drug in question. In regard to the commercial availability of the pharmaceuticals of the present disclosure: ERT3 is available and is generally compounded for extended release formulation by compounding pharmacies; PDE 5 inhibitors are available and FDA approved in the form of sildenafil, tadalafil, vardenafil and avanafil. Presently there are no oral PDE 3/4 inhibitors which are FDA approved. Tolafentrine and pumafentrine were abandoned some years ago following unsatisfactory trials. Should any new PDE 3/4 inhibitors be developed successfully in the tolafentrine/pumafentrine lineage, they would be candidates for use in the art of the present disclosure. Cilostazol is a PDE 3 inhibitor presently FDA approved for peripheral vascular disease. Roflumilast is a PDE 4 inhibitor FDA approved for chronic obstructive lung disease.

The pharmaceutical and dosing interval for example 1 comprises, without exclusion or limitation, the following: ERT3 1-9 mcg (with or without T4 10-30 mcg) dosed every 12 hours. The polypharmaceutical and dosing interval for example 2 comprises, without exclusion or limitation, the following:

1. ERT3 1-9 mcg (with or without T4 10-30 mcg).

2. A PDE 3/4 inhibitor, or separate PDE 3 and PDE 4 inhibitors.

3. A PDE 5 inhibitor.

Elements 1-3 are dosed every 12 hours.

Application of example 2; 2 is limited at the time of writing by the fact that combined PDE 3/4 inhibitors are only available for experimental purposes, generally for use by inhalation. Until there is FDA approval of a new PDE 3/4 inhibitor, separate PDE 3 and PDE 4 inhibitors would have to be used for the present disclosure, or ERT3 and a PDE 5 inhibitor could be used together without a PDE 3/4 inhibitor. Details on the abandonment of the tolafentrine clinical trials are not available. In the event that they were abandoned for reasons of lack of efficacy and not for patient safety issues, tolafentrine could be resurrected and studied experimentally as part of the method of the present disclosure. Practical application of example 2; 3 is not limited. The PDE 5 inhibitors sildenafil, tadalafil and vardenafil and avanafil are all FDA approved and in clinical use.

A deliberate attempt has been made to fit the theory of imprinted genes into the present disclosure. This may, at times, appear awkward. Further, those skilled in the art will note inconsistencies, chiefly that the PIG does not have control over the outcome, which may result in the death of the fetus. Notwithstanding these issues, the veracity (or lack thereof) of the PIG hypothesis of PE has no bearing whatsoever on the validity of the pathophysiology of PE described in the disclosure or the merits of the therapeutic intervention of the method of the disclosure. There is solid basis for the delineated PE pathophysiology, which is well supported by the literature in references. Further, there is a solid pharmacologic basis for the method of the present disclosure which is also well supported by the literature in references.

It should be noted that the examples described above are provided for purposes of illustration, and are not intended to be limiting. Other formulations, dosing regimens, and devices and/or device configurations may be utilized to carry out the disclosure described herein. It can be envisioned that technology advances in the field may lead to variations of the disclosure.

### REFERENCES

1. Gereben B, et al. 2008: Cellular and Molecular Basis of Deiodinase-Regulated Thyroid Hormone Signaling. Endoc. Rev. December; 29(7) 898-938.
2. Arroyo e Drigo R, et al. 2011: Endoplasmic Reticulum Stress Decreases Intracellular Thyroid Hormone Activation via an e1F2a-Mediated Decrease in Type 2 Deiodinase Synthesis. Molec. Endocrinol. December. 25(12) 2065-75
3. Dentice M, Salvatore D 2011: Deiodinases: the balance of thyroid hormone: Local impact of thyroid hormone inactivation. J. Endocrinol. 209(3): 273-282
4. Phipps E A, et al. 2019: Pre-eclampsia: pathogenesis, novel diagnostics and therapies. Nature Rev. Nephrol. 15(5): 275-89
5. Ahmed A, Ramma W 2015: Unravelling the theories of pre-eclampsia: are the protective pathways the new paradigm? Brit. J. Pharmacology 172(6) 1574-86
6. Venditti C C, Smith G N 2014: Involvement of the heme oxygenase system in the development of pre-eclampsia and as a possible therapeutic target. Womens Health 10(6); 623-43
7. Gumaa K A, et al 1977: Thyroid Hormone Control of Cyclic Nucleotide Phosphodiesterases and the Regulation of the Sensitivity of the Liver to Hormones. FEBS LETTERS 80(1); 45-8.
8. Van Inwegen R G, et al. 1975: Cyclic Nucleotide Phosphodiesterases and Thyroid Hormones. J. Biol. Chem. 250(7) 2452-56
9. Ferguson-Smith A, Bourc'his D 2018: The discovery and importance of genomic imprinting. eLIFE; doi.org/10.7554/eLIFE.42368
10. Liao Q P, et al. 2019: SHH expression in placental tissues and trophoblastic cell oxidative stress injury during pre-eclampsia. Eur Rev Med. and Pharmacol Sci 23(14): 6026-34
11. Alam S et al. 2017: Role of TGF-B1 in the Pathogenesis of Pre-Eclampsia. Ann. Int. Med. Dent. Research 3(3) 1-4
12. Huang S A, et al 2005: Transforming growth factor-beta promotes inactivation of extracellular thyroid hormones via transcriptional stimulation of type3 iodothyronine deiodinase. Mol. Endocrinol. December; 19(12): 3126-36
13. Basbug M, et al 1999: Correlation between maternal thyroid function tests and endothelin in preeclampsia-eclampsia. Obstet. Gynecol. 94(4): 551-5
14. Yuan J, et al 2017: Circulating asymmetric dimethylarginine and the risk of preeclampsia: a meta-analysis based on 1338 participants. Oncotarget Vol. 8 (27) 43944-43952
15. Vadillo-Ortega F, et al 2011: Effect of supplementation during pregnancy with L-arginine and antioxidant vitamins in medical food on pre-eclampsia in high risk population: randomized controlled trial. BMJ 342:d2901
16. Novak P, Soukop T 2011: Calsequestrin Distribution, Structure and Function, Its role in Normal and Pathological Situations and the Effect of Thyroid Hormones. Physiological Research/Academia Scientiarum Bohemoslovaca 60(3): 439-52
17. Ojamaa K, et al 2000: Thyroid Hormone Regulation of Phospholamban Phyosphorylation in the Rat Heart. Endocrinology 141(6) 2139-44
18. Serfass L, et al 2001: Calcium Ion Downregulates Soluble Guanyl Cyclase Activity: Evidence for a Two-metal Ion Catalytic Mechanism. Arch. Biochem. Biophys. 387(1) 47-56
19. Quesada A, et al 2002: Nitric oxide synthase activity in hyperthyroid and hypothyroid rats. Eur. J. Endocrinol. 147; 117-22
20. Li F, et al 2011: Heme oxygenase-1 is induced by thyroid hormone and involved in thyroid hormone preconditioning-induced protection against renal warm ischemia in rat. Mol Cell Endocrinol. 339(1-2) 54-62
21. Pullamsetti S S, et al 2011: cAMPphosphodiesterase inhibitors increases nitric oxide production by modulating dimethylarginine dimethylaminohydrolases. Circulation 123(11) 1194-1204
22. Simon-Tillaux N, et al 2019: Sildenafil for the treatment of preeclampsia, an update: should we still be enthusiastic? Nephrol Dial Transplant 34(11): 1819-26
23. Walsh M 2006: Laboratory Procedure Manual: Free T3. University of Washington Medical Center, Dept. of Laboratory Medicine.

What is claimed is:

1. A method for preventing or treating pre-eclampsia (PE) in a pregnant human patient, the method comprising the steps of: a) providing L-tri-iodothyronine (T3) in a extended release formulation; and b) administering said extended release formulation to the pregnant human patient.

2. The method of claim 1 further comprising the step of providing L-thyroxine (T4) in the formulation.

3. The method of claim 1, wherein the T3 is provided in the extended release formulation at a dose of at least 2 mcg per 24 hours.

4. The method of claim 1, wherein the T3 is provided in the extended release formulation at a dose not more than 18 mcg per 24 hours.

5. The method of claim 1, wherein administration of the formulation occurs before 12 weeks of gestation.

6. The method of claim 1, wherein administration of the formulation occurs after 12 weeks of gestation.

7. The method of claim 1, wherein administration of the formulation begins at the time a diagnosis of pre-eclampsia (PE) is made.

8. The method of claim 1 further comprising including at least one phosphodiesterase (PDE) inhibitor in the formulation.

9. A composition for prevention or treatment of pre-eclampsia (PE), the composition comprising extended release L-tri-iodothyronine (T3) and at least one phosphodiesterase (PDE) inhibitor.

10. The composition of claim 9 further comprising L-thyroxine (T4).

11. The composition of claim 9, wherein T3 is present in a dose of at least 2 mcg.

12. The composition of claim 9, wherein T3 is present in a dose of not more than 18 mcg.

13. The composition of claim 9, wherein at least one PDE inhibitor is a PDE 3/4 inhibitor.

14. The composition of claim 9, wherein the at least one PDE inhibitor is a PDE 5 inhibitor.

15. A composition for prevention or treatment of pre-eclampsia (PE) the composition comprising controlled release L-tri-iodothyronine (T3), L-thyroxine (T4), and at least one phosphodiesterase (PDE) inhibitor.

16. The composition of claim 15, wherein the PDE inhibitor is a PDE type 3/4 inhibitor.

17. The composition of claim 15, wherein the PDE inhibitor is a PDE type 5 inhibitor.
